(19) United States
(12) Patent Application Publication
(10) Pub. No.: US 2021/0308266 A1
(43) Pub. Date: Oct. 7, 2021

(54) LIPOFULLERENE CONJUGATES AND THEIR USE FOR PROMOTION OF HAIR GROWTH AND PREVENTION OF HAIR LOSS

(71) Applicant: LivePet, LLC, Austin, TX (US)

(72) Inventors: Ian Mitchell, Bartlesville, OK (US); Daniel David Bensimon, Austin, TX (US)

(21) Appl. No.: 17/270,353

(22) PCT Filed: Aug. 19, 2019
(86) PCT No.: PCT/US2019/047057

§ 371 Date: Feb. 22, 2021

Related U.S. Application Data
(60) Provisional application No. 62/719,951, filed on Aug. 20, 2018.

(51) Int. Cl. A61K 47/54; A61K 33/44; A61K 9/00

(52) U.S. Cl. CPC A61K 47/542; A61K 9/0019; A61K 33/44

(57) ABSTRACT

In some embodiments, a system and/or method may include a chemical composition which functions to promote hair growth and/or inhibits hair loss. The chemical composition may include a lipofullerene. The chemical composition may include activator. The activator may include a physiologically acceptable carrier. The lipofullerene may be applied to a subject separately from the activator. The lipofullerene may be applied to the subject first and the activator may be applied to the subject after in the same general area of the subject.

### BACKGROUND OF THE INVENTION

### 1. Field of the Invention

The present disclosure generally relates to pharmaceutically acceptable formulations for promoting hair growth and/or preventing hair loss. More particularly, the disclosure generally relates to a pharmaceutically acceptable formulation comprising a lipofullerene conjugate for promoting hair growth and/or preventing hair loss.

### 2. Description of the Relevant Art

A major cause of hair loss is typically attributed to genetic factors. More recently, it has been argued hair loss among the general population has gradually increased due to environmental pollution and poor eating habits (e.g., processed foods) in addition to an increase in social stress.

Hair is maintained by a repeating hair growth and hair loss cycle of anagen, catagen and telogen. Anagen is the growth phase. Catagen is the involuting or regressing phase. Telogen is the resting or quiescent phase. Each phase has several morphologically and histologically distinguishable sub-phases. Prior to the start of cycling is a phase of follicular morphogenesis. There is also a shedding phase, or exogen, that is independent of anagen and telogen in which one or several hairs that might arise from a single follicle exits.

Hair loss is a normal phenomenon. However normal people have a large number of hairs in an anagen state, and on the contrary, people with alopecia have a large number of hairs in the telogen phase, such that a hair loss phenomenon is visually noticeable.

People with alopecia are characterized by miniaturization of hair. As hair loss proceeds, a period of the anagen is reduced, and accordingly, the miniaturization of hair also proceeds. Therefore, in order to treat hair loss, it is important to induce hair follicles from the telogen phase into the anagen phase quickly, and to increase the anagen phase.

Male pattern alopecia is a phenomenon caused by the male hormone testosterone. When this testosterone is changed into stronger dihydrotestosterone (DHT) by 5 α-reductase, this hormone acts on the hair follicles by inducing the hair follicles from the anagen into the catagen, resulting in hair loss. Therefore, in order to treat alopecia caused by the above reason, a method of inhibiting production of DHT by 5 α-reductase is mainly used.

Female pattern alopecia is mainly caused by reduction of an amount of estrogen after menopause. In the female pattern alopecia, hair loss does not occur in a front part of a head, but mainly occurs in a middle part of a head. The effect of 5 α-reductase on the female pattern alopecia is smaller than that on the male pattern alopecia. Therefore, drugs inhibiting 5 α-reductase are not significantly effective for women with alopecia after menopause. Accordingly, minoxidil or estrogen is primarily used as therapeutic agents for alopecia.

Alopecia areata is caused by autoimmune diseases, mental stress, or genetic predisposition. A cause of the alopecia areata is fundamentally different from that of androgenetic alopecia, and a treatment method thereof is also different from that of androgenetic alopecia.

Currently known hair loss prevention products include components that aim at promoting blood circulation, inhibiting male hormone action, enhancing hair root function, etc. However, current products do not appear to have any appreciable effect and many have side effects.

The present disclosure relates to compositions based at least in part on fullerenes. Since the discovery of fullerenes (Carbon 60 (C60)) in 1985, fullerenes have triggered interest in the scientific community based upon fullerenes' interesting properties. Fullerene has been found to comprise a number of desirable structural, physical, and chemical properties adaptable for biological uses including antioxidants, anti-inflammatory, drug delivery, and photodynamic therapy.

Because of their spherical shape, structural similarity and their tilde one nanometer size, fullerenes are often denoted as buckyballs or carbon nanomaterials. C60 has a uniquely delocalized π electron cloud across the cage surface with each carbon atom contributing one π electron, rendering fullerene with very high electron affinity. The absence of any reactive site on the cage surface except the carbon-carbon double bonds often makes the cage inert under physiological conditions. From the chemistry perspective, fullerenes are super-conjugated, electron deficient poly-olefins. From the biology perspective, fullerenes are super-powerful antioxidants capable of scavenging and detoxifying reactive oxygen species (ROS) and reactive nitrogen species (RNS).

Various functionalized fullerenes were synthesized through chemical or supra-molecular approaches, usually a chemical modification on the surface of the sphere, to achieve promising results. Most likely due to its unique chemical structure, C60 possesses interesting photo-physical properties and generates reactive oxygen species by exposure to visible light, making it a potentially strong agent for photodynamic therapy in biological systems. Fullerenes can efficiently generate reactive oxygen species when exposed to visible light, which means it may be an effective cytotoxic agent.

Aqueous suspensions were previously used to investigate the acute and sub-acute toxicities as well as the in vivo antioxidant properties of C60. When taken in-vivo, C60 complexes resulted in varying degree of toxicity. But, such suspensions are not appropriate for determining toxicity at reiterated doses, because fullerene is active only in soluble form and because the extremely slow dissolution of C60 in biological media prevents controlling accurately the active fraction.

Therefore, a composition for promoting hair growth and/or preventing hair loss would be highly desirable.

### SUMMARY

In some embodiments, a system and/or method may include a chemical composition which functions to promote hair growth and/or inhibits hair loss. The chemical composition may include a lipofullerene. The chemical composition may include activator. The activator may include a physiologically acceptable carrier. The lipofullerene may be applied to a subject separately from the activator. The lipofullerene may be applied to the subject first and the activator may be applied to the subject after in the same general area of the subject.

In some embodiments, the lipofullerene may be based on the fullerene C60. The lipofullerene may be formed by reacting a lipid with a fullerene. The lipid may include a fatty acid. The lipid may include a structural formula of C8H16O2. The lipid may include a short to medium chain length fatty acid (e.g., up to C12). The lipofullerene comprises a structural formula of [C60]x[C8H14O2]y, wherein x is greater than or equal to 1, and wherein y is greater than or equal to 1.

In some embodiments, the activator further includes a solvent which functions as a diluent. The activator may further include water as the solvent. The physiologically acceptable carrier may include a polar solvent. The physiologically acceptable carrier may include a nontoxic polar solvent.

In some embodiments, the chemical compound may be incorporated in a pharmaceutical composition.

In some embodiments, a method may include promoting hair growth and/or inhibiting hair loss. The method may include topically administering to a subject an effective amount of a chemical compound including a lipofullerene. The method may include topically administering to the subject an activator. The method may include promoting hair growth and/or inhibiting hair loss.

In some embodiments, a method may include affecting the cellular permeability using the activator. The method may include binding at least a portion of the activator to the lipofullerene to convey the lipofullerene intracellularly. The method may include affecting (e.g., increasing) production of adenosine triphosphate (ATP) and making the electron transport chain (ETC) more efficient.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts a proposed structure of an embodiment of a lipofullerene conjugate.

FIGS. 2A-C depict photographs of before and after pictures of a subject whom has applied the compositions described herein to promote hair growth.

FIGS. 3A-D depict photographs of before and after pictures of a subject whom has applied the compositions described herein to promote hair growth.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task. In some contexts, “configured to” may be a broad recitation of structure generally meaning “having a feature that” performs the task or tasks during operation. As such, the component can be configured to perform the task even when the component is not currently on.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION

### Definitions

Unless defined otherwise, all technical and scientific terms used herein have the same meaning as commonly understood by one of ordinary skill in the art.

The terms “administration,” “administering,” or the like, as used herein when used in the context of providing a pharmaceutical, cosmeceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

The term “animal” as used herein generally refers to any member of the kingdom Animalia, comprising multicellular organisms that have a well-defined shape and usually limited growth, can move voluntarily, actively acquire food and digest it internally, and have sensory and nervous systems that allow them to respond rapidly to stimuli: some classification schemes also include protozoa and certain other single-celled eukaryotes that have motility and animal like nutritional modes. Generally, the term animal as used herein does not refer to humans.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

The terms “effective concentration” or “effective amount” as used herein generally refers to a sufficient amount of the pharmaceutically active agent that is added to decrease, promote hair growth and/or prevent hair loss. The amount will vary for each compound and upon known factors related to the item or use to which the pharmaceutically active agent is applied.

The term “fullerene” as used herein generally refers to is a molecule of carbon in the form of a hollow sphere, ellipsoid, tube, and many other shapes.

The terms “in need of treatment” or “in need thereof” when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

The term “lipid” as used herein generally refers to a substance of biological origin that is soluble in nonpolar solvents or more generally as hydrophobic or amphiphilic small molecules. Specific examples of lipids include fats, waxes, sterols, fat-soluble vitamins (e.g., vitamins A, D, E, and K), monoglycerides, diglycerides, triglycerides, and phospholipids.

The term “malady” as used herein generally refers to any disorder or disease of the body or any undesirable or disordered condition including, but not limited to, illness, sickness, affliction, complaint, ailment, indisposition, virus, disease, fungus, infection, disease, etc.

The term “mammal” as used herein generally refers to any vertebrate of the class Mammalia, having the body more or less covered with hair, nourishing the young with milk from the mammary glands, and, with the exception of the egg-laying monotremes, giving birth to live young. Generally, the term mammal as used herein does not refer to humans.

The terms “oligomeric” and “polymeric” as used herein are generally used interchangeably herein to generally refer to multimeric structures having more than one component monomer or subunit.

Terms such as “pharmaceutical composition,” “pharmaceutical formulation,” “pharmaceutical preparation,” or the like, are used herein to generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release,” “controlled release,” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

A “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

The term “pharmacologically inert,” as used herein, generally refers to a compound, additive, binder, vehicle, and the like, that is substantially free of any pharmacologic or “drug-like” activity.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “subject” as used herein generally refers to a mammal (e.g., felines, canines), and in particular to a human.

The phrase “therapeutically effective amount” generally refers to an amount of a drug or pharmaceutical composition that will elicit at least one desired biological or physiological response of a cell, a tissue, a system, animal or human that is being sought by a researcher, veterinarian, physician or other caregiver.

### Embodiments

Accordingly, there exists a need for an improved composition which promotes hair growth and/or inhibits hair loss.

In some embodiments, compositions described herein revive hair growth by targeting hair follicles and restarting a subject's hair growth cycle. Compositions described herein may reactivate hair follicles that have stalled due to hereditary hair loss, physical conditions, or environmental factors that may affect follicular production. Compositions described herein may help subjects grow thicker, fuller hair.

In some embodiments, compositions described herein penetrate a subject's scalp and reaches the follicular cells where it rejuvenates inactive hair follicles and increases intracellular energy production to kickstart your hair's anagen growth phase. Visible results may occur within 3 to 6 weeks.

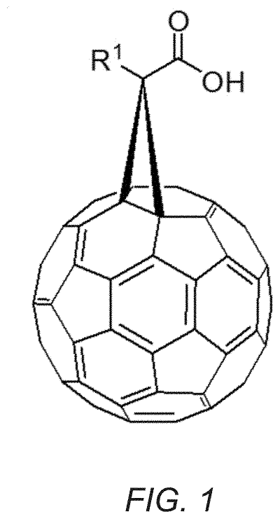

In some embodiments, a system and/or method may include a chemical composition which functions to promote hair growth and/or inhibits hair loss. The chemical composition may include a lipofullerene. The chemical composition may include activator. The activator may include a physiologically acceptable carrier. The lipofullerene may be applied to a subject separately from the activator. The lipofullerene may be applied to the subject first and the activator may be applied to the subject after in the same general area of the subject. FIG. 1 depicts an embodiment of a lipofullerene conjugate.

In some embodiments, the lipofullerene may be based on the fullerene C60. Fullerenes may include molecules of carbon in the form of a hollow sphere, ellipsoid, tube, and many other shapes. A fullerene is a molecule of carbon in the form of a hollow sphere, ellipsoid, tube, and many other shapes. Spherical fullerenes may be referred to as buckminsterfullerene or buckyballs or C60, and they resemble the balls used in soccer. Cylindrical fullerenes may be referred to as carbon nanotubes or buckytubes. Fullerenes are similar in structure to graphite, which is composed of stacked graphene sheets of linked hexagonal rings; but they may also contain pentagonal (or sometimes heptagonal) rings. Fullerenes are stable, but not unreactive. The sp²-hybridized carbon atoms must be bent to form the closed sphere or tube, which produces angle strain. The characteristic reaction of fullerenes is electrophilic addition at 6,6-double bonds, which reduces angle strain by changing sp²-hybridized carbons into spa-hybridized ones. The change in hybridized orbitals causes the bond angles to decrease from about 120° in the sp² orbitals to about 109.5° in the sp³ orbitals. This decrease in bond angles allows for the bonds to bend less when closing the sphere or tube, and thus, the molecule becomes more stable.

Fullerenes have been used for several biomedical applications including the design of high-performance MRI contrast agents, X-Ray imaging contrast agents, photodynamic therapy and drug and gene delivery. Research on fullerene toxicity beginning in the early 1990s to present appears to conclude that very little evidence gathered since the discovery of fullerenes indicates that C60 is toxic. The toxicity of these carbon nanoparticles appears to be not only dose and time-dependent, but also depends on a number of other factors such as: type (e.g., C60, C70, functional groups used to water solubilize these nanoparticles (e.g., OH, COOH), and method of administration (e.g., intravenous, intraperitoneal)).

The lipofullerene may be formed by reacting a lipid with a fullerene. The lipid may include a fatty acid (e.g., caprylic acid). The lipid may include a structural formula of C8H16O2. The lipid may include a short to medium chain length fatty acid (e.g., up to C12). The lipofullerene comprises a structural formula of [C60]x[C8H14O2]y, wherein x is greater than or equal to 1, and wherein y is greater than or equal to 1.

In some embodiments, the lipid may include a fatty acid. A lipid may refer to a substance of biological origin that is soluble in nonpolar solvents or more generally as hydrophobic or amphiphilic small molecules. Specific examples of lipids may include fats, waxes, sterols, fat-soluble vitamins (e.g., vitamins A, D, E, and K), monoglycerides, diglycerides, triglycerides, and phospholipids. The main biological functions of lipids include storing energy, signaling, and acting as structural components of cell membranes. In some embodiments, the lipid may include a structural formula of C8H16O2. Other lipids have been examined but the current fatty acid had a more pronounced beneficial effect.

Lipids may be broadly defined as hydrophobic or amphiphilic small molecules. The amphiphilic nature of some lipids may allow them to form structures such as vesicles, multilamellar/unilamellar liposomes, or membranes in an aqueous environment. Biological lipids originate entirely or in part from two distinct types of biochemical subunits or “building-blocks” ketoacyl and isoprene groups. Using this approach, lipids may be divided into eight categories: fatty acids, glycerolipids, glycerophospholipids, sphingolipids, saccharolipids, polyketides, sterol lipids and prenol lipids.

Lipids may encompass molecules such as fatty acids and their derivatives (including tri-, di-, monoglycerides, and phospholipids), as well as other sterol-containing metabolites such as cholesterol. Although humans and other mammals use various biosynthetic pathways to both break down and synthesize lipids, some essential lipids cannot be made this way and must be obtained from the diet.

In some embodiments, the activator further includes a solvent which functions as a diluent. The activator may further include water as the solvent. The physiologically acceptable carrier may include a polar solvent. The physiologically acceptable carrier may include a nontoxic polar solvent. The physiologically acceptable carrier may include dimethyl sulfoxide (DMSO). The physiologically acceptable carrier may include methylsulfonylmethane (MSM). In some embodiments, DMSO may be used and diluted with a solvent (e.g., water). DMSO may be diluted to about 30-70%, 40-60%, or about 50%.

In some embodiments, the chemical compound may be incorporated in a pharmaceutical composition.

In some embodiments, a method may include promoting hair growth and/or inhibiting hair loss. The method may include topically administering to a subject an effective amount of a chemical compound including a lipofullerene. The method may include topically administering to the subject an activator. The method may include promoting hair growth and/or inhibiting hair loss. The compounds may be applied topically due to the general lack of blood flow in the scalp not allowing for ingested compounds to be transported via a subject's bloodstream.

In some embodiments, a method may include affecting the cellular permeability using the activator. The method may include binding at least a portion of the activator to the lipofullerene to convey the lipofullerene intracellularly. The method may include affecting (e.g., increasing) production of adenosine triphosphate (ATP) and making the electron transport chain (ETC) more efficient.

Compositions described herein used as directed (e.g., daily) may revive and maintain hair regrowth. In some embodiments, before starting use of compositions described herein, stop use of all hair products (including shampoos and conditioners) for 2 days.

In some embodiments, compositions described herein may be applied nightly before going to bed to activate hair regrowth. Visible hair growth may be seen within a few weeks. In some embodiments, a daily routine using compositions described herein on desired area of scalp to maintain hair regrowth must be continued.

In some embodiments, compositions described herein may be applied by first cleaning area where subject will be applying lipofullerene with warm water and wipe dry. The subject then may gently scratch both the area on the scalp that subject wants to regrow and the area directly adjacent to it (where the hair is currently growing). This may be done for 30 to 45 seconds across the desired area to provide adequate stimulation. Next apply 3 to 10 drops of the lipofullerene depending on the size of the area where you massaged your scalp (and the concentration). Gently massage the lipofullerenes into the same area of the scalp and adjacent hair (e.g., for 1 minute). Side effects may include tingly sensation and reddening of the skin.

In some embodiments, an activator is then applied the skin area (e.g., 3-10 drops). The skin may be again massaged (e.g., for 20-30 seconds).

If the sensation is too tingly or intense, more of the lipofullerene may be rubbed in to dissipate the activator solution. If you have put too much of the activator solution on, wash some of it off by putting drops of cold water on the desired area and use a clean towel to remove a bit via gentle rubbing of the scalp.

In some embodiments, the compositions are left on scalp for 60 minutes or overnight.

In some embodiments, after a period of time (e.g., 60 minutes) the compositions are removed with warm water from the skin. Shampoo, soap, or other products may not be used to remove and should not be used for a period of time (e.g., about 2 hours). Inadvertently applied compositions may be removed with a water using an applicator.

In some embodiments, the chemical compound is incorporated in a pharmaceutical or chemical composition. In some embodiments, the lipofullerene may be formed using at least some ionic bonds. In some embodiments, the lipofullerene may be formed using covalent bonds. In some embodiments, the lipid and fullerene may form an adduct. The term “adduct” as used herein generally refers to a product of a direct addition of two or more distinct molecules, resulting in a single reaction product containing all atoms of all components. In some embodiments, the lipid and fullerene may form an ionic bond. In some embodiments, the lipid and fullerene may form a covalently bound compound.

### Dosage and Administration

In some embodiments, chemical compositions described herein may be administered at a dosage level up to conventional dosage levels, but will typically be less than about 50 mL per day. Suitable dosage levels for chemical compositions described herein may be about 0.01 mg to 10 mg per kg body weight of the patient per day, from about 0.1 mg to 1 mg per kg body weight of the patient per day, or from about 0.01 mg to 0.1 mg per kg body weight of the patient per day. The compound may be administered on a regimen of up to 6 times per day, between about 1 to 4 times per day, or once per day.

In the case where an oral composition is employed, a suitable dosage range is, e.g. from about 0.01 mg to about 10 mg per kg of body weight per day, preferably from about 0.1 mg to about 0.5 mg per kg.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

In addition to administering chemical compositions described herein as described, the compounds may be administered as part of a pharmaceutical preparation containing suitable pharmaceutically acceptable carriers, preservatives, excipients and auxiliaries which facilitate processing of the chemical compositions described herein which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

### Pharmaceutical Compositions

Chemical compositions described herein are typically administered topically but any suitable route of administration may be employed for providing a subject with an effective dosage of drugs of the chemical compositions described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulizers. The compounds may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, gels, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives. Optionally, the resulting mixture may be ground and processed. The resulting mixture of granules may be used, after adding suitable auxiliaries, if desired or necessary, to obtain tablets, softgels, lozenges, capsules, or dragee cores.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

Liquid dosage forms for oral administration can contain coloring and flavoring to increase subject acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

In some embodiments, an oral composition may include a flavoring. A flavoring may include something an animal may find palatable. For example a flavoring may include malt extract, xylitol, splenda, sucralose or any sweetener. A flavoring may range from 0.01% to 0.10%, 0.10% to 1.0%, or 1.0% to 10.0% of a composition.

In some embodiments, a composition may include a colorant. A colorant may include D&C Blue #1 or any FDA approved color. A colorant may range from 0.001% to 0.010%, 0.010% to 0.10%, or 0.10% to 1.0% of a composition.

Additional oral compositions which may be used to deliver chemical compositions described herein, as well as additional uses, are described in U.S. Pat. No. 4,666,896 to Warner, Jr. et al., U.S. Pat. No. 5,393,516 to Rheinberger et al., and U.S. Pat. No. 5,948,390 to Nelson et al., as well as U.S. Patent Publication No. 2005/0158252 to Romanowski et al., which are incorporated by reference as if fully set forth herein.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas. Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Liposomal formulations, in which mixtures of the chemical compositions described herein with, for example, egg yolk phosphotidylcholine (E-PC), may be made for injection. Optionally, the suspension may contain stabilizers, for example, antioxidants such as BHT, and/or preservatives, such as benzyl alcohol.

In some embodiments, an oral composition may include a fragrance.

In some embodiments, a composition may include additional additives which may function in combination or separately from the chemical compositions described herein in solution. Additives may function to improve a subject's health. Additives may include vitamins including, but not limited to, vitamins D and E.

In some embodiments, different compositions may be formulated for different types of users. For professionals users (e.g., doctors, veterinaries), compositions may include a greater percentage of chemical compositions described herein than compositions formulated for over the counter sale to nonprofessionals. Professional compositions may not include flavorings or colorants.
CLAUSE A: A chemical composition, comprising a lipofullerene; and an activator comprising a physiologically acceptable carrier.

The chemical composition of CLAUSE A, wherein the fullerene comprises C60.

The chemical composition of CLAUSE A or any one of the paragraphs dependent on CLAUSE A, wherein the lipofullerene is formed by reacting a lipid with a fullerene.

The chemical composition of CLAUSE A or any one of the paragraphs dependent on CLAUSE A, wherein the activator further comprises a solvent which functions as a diluent.

The chemical composition of CLAUSE A or any one of the paragraphs dependent on CLAUSE A, wherein the activator further comprises water.

The chemical composition of CLAUSE A or any one of the paragraphs dependent on CLAUSE A, wherein the physiologically acceptable carrier comprises a polar solvent.

The chemical composition of CLAUSE A or any one of the paragraphs dependent on CLAUSE A, wherein the physiologically acceptable carrier comprises a nontoxic polar solvent.

The chemical composition of CLAUSE A or any one of the paragraphs dependent on CLAUSE A, wherein the lipofullerene is formed by reacting a lipid with a fullerene, and wherein the lipid comprises a fatty acid.

The chemical composition of CLAUSE A or any one of the paragraphs dependent on CLAUSE A, wherein the lipofullerene is formed by reacting a lipid with a fullerene, and wherein the lipid comprises a structural formula of C8H16O2.

The chemical composition of CLAUSE A or any one of the paragraphs dependent on CLAUSE A, wherein the lipofullerene is formed by reacting a lipid with a fullerene, and wherein the lipid comprises a short to medium chain length fatty acid.

The chemical composition of CLAUSE A or any one of the paragraphs dependent on CLAUSE A, wherein the lipofullerene is formed by reacting a lipid with a fullerene, and wherein the lipid comprises a short to medium chain length fatty acid comprising up to C12.

The chemical composition of CLAUSE A or any one of the paragraphs dependent on CLAUSE A, wherein the lipofullerene comprises a structural formula of [C60]x[C8H14O2]y, wherein x is greater than or equal to 1, and wherein y is greater than or equal to 1.

The chemical composition of CLAUSE A or any one of the paragraphs dependent on CLAUSE A, wherein the chemical composition is incorporated in a pharmaceutical composition.
CLAUSE B: A method of promoting hair growth and/or inhibiting hair loss, comprising: topically administering to a subject an effective amount of a chemical compound comprising a lipofullerene; topically administering to the subject an activator; and promoting hair growth and/or inhibiting hair loss.

The method of CLAUSE B or any one of the paragraphs dependent on CLAUSE B, further comprising affecting the cellular permeability using the activator.

The method of CLAUSE B or any one of the paragraphs dependent on CLAUSE B, further comprising binding at least a portion of the activator to the lipofullerene to convey the lipofullerene intracellularly.

The method of CLAUSE B or any one of the paragraphs dependent on CLAUSE B, further comprising increasing production of ATP.

CLAUSE B: A chemical composition for use in promoting hair growth and/or inhibiting hair loss, comprising: topically administering to a subject an effective amount of a chemical compound comprising a lipofullerene; topically administering to the subject an activator; and promoting hair growth and/or inhibiting hair loss.

The chemical composition of CLAUSE C or any one of the paragraphs dependent on CLAUSE C, further comprising affecting the cellular permeability using the activator.

The chemical composition of CLAUSE C or any one of the paragraphs dependent on CLAUSE C, further comprising binding at least a portion of the activator to the lipofullerene to convey the lipofullerene intracellularly.

The chemical composition of CLAUSE C or any one of the paragraphs dependent on CLAUSE C, further comprising increasing production of ATP.

A chemical composition for use in promoting hair growth and/or inhibiting hair loss.

### Examples

Having now described the invention, the same will be more readily understood through reference to the following example(s), which are provided by way of illustration, and are not intended to be limiting of the present invention.

In some embodiments, buckminsterfullerene (C60) is bound with a lipid base.

### Anecdotal Evidence

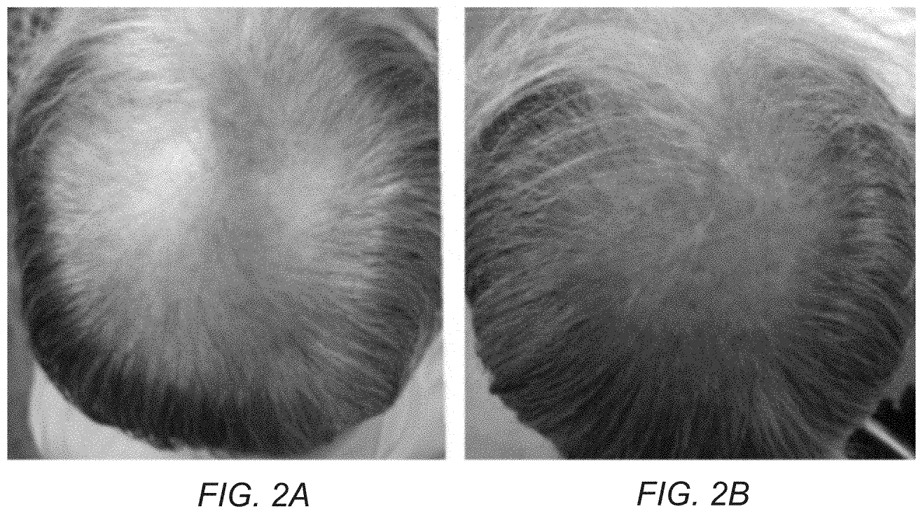

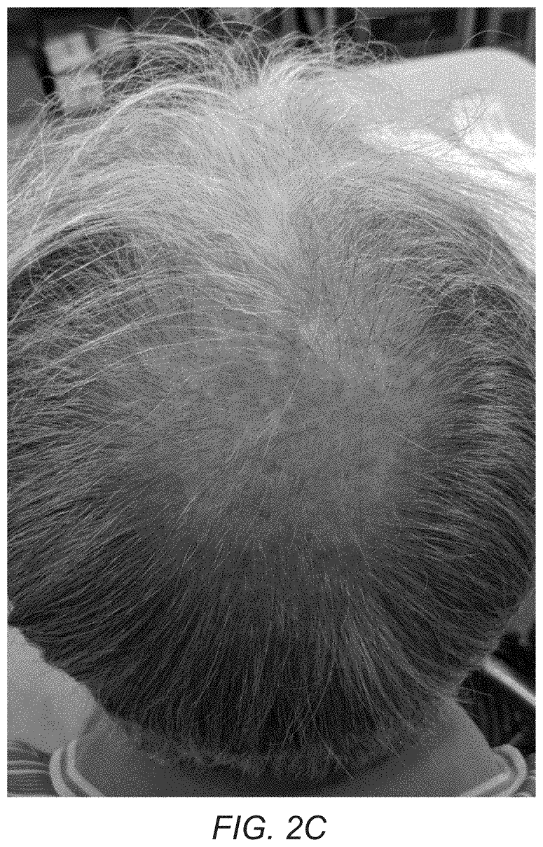

FIGS. 2A-C depict photographs of before (FIG. 2A) and after (FIGS. 2B-C) pictures of a subject whom has applied the compositions described herein to promote hair growth.

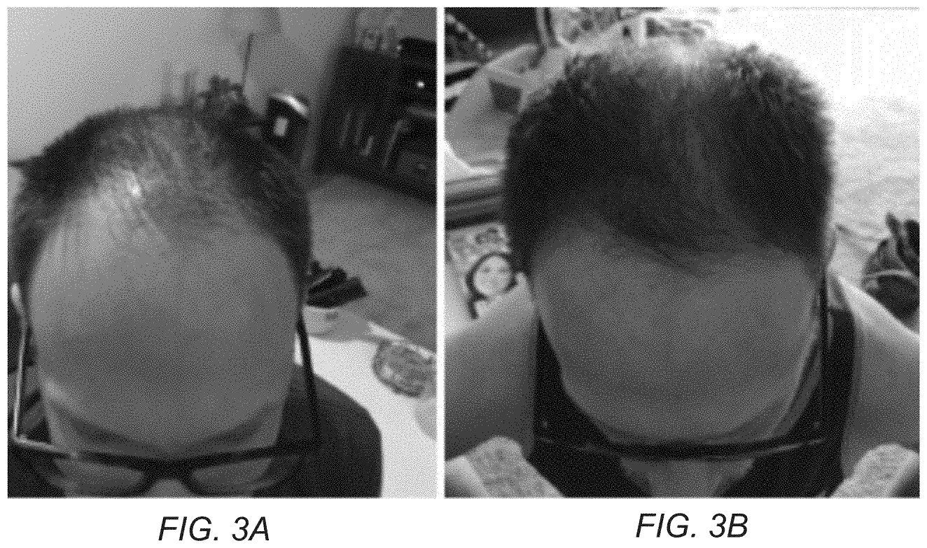

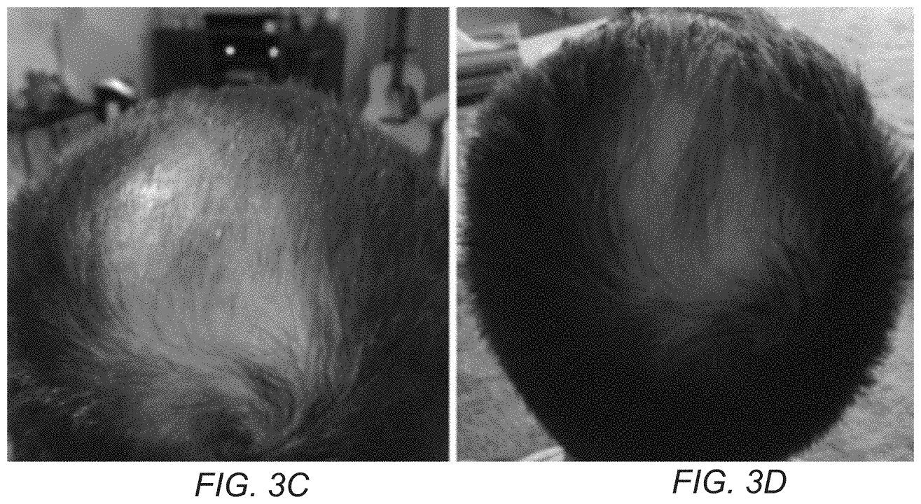

FIGS. 3A-D depict photographs of before (FIGS. 3A and C) and after (FIGS. 3B- and D) pictures of a subject whom has applied the compositions described herein to promote hair growth.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A chemical composition for topical application for use in promoting hair growth and/or inhibiting hair loss, comprising:
a lipofullerene; and
an activator comprising a physiologically acceptable carrier to promote transdermal intracellular uptake of the lipofullerene.

2. The chemical composition of claim 1, wherein the fullerene comprises C60.

3. The chemical composition of claim 1, wherein the lipofullerene is formed by reacting a lipid with a fullerene.

4. The chemical composition of claim 1, wherein the activator further comprises a solvent which functions as a diluent for the activator.

5. The chemical composition of claim 1, wherein the activator further comprises a solvent which functions as a diluent for the activator, and wherein the solvent comprises water.

6. The chemical composition of claim 1, wherein the physiologically acceptable carrier comprises a polar solvent.

7. The chemical composition of claim 1, wherein the physiologically acceptable carrier comprises a nontoxic polar solvent.

8. The chemical composition of claim 1, wherein the lipofullerene is formed by reacting a lipid with a fullerene, and wherein the lipid comprises a fatty acid.

9. The chemical composition of claim 1, wherein the lipofullerene is formed by reacting a lipid with a fullerene, and wherein the lipid comprises a structural formula of C8H16O2.

10. The chemical composition of claim 1, wherein the lipofullerene is formed by reacting a lipid with a fullerene, and wherein the lipid comprises a short to medium chain length fatty acid.

11. The chemical composition of claim 1, wherein the lipofullerene is formed by reacting a lipid with a fullerene, and wherein the lipid comprises a short to medium chain length fatty acid comprising up to C12.

12. The chemical composition of claim 1, wherein the lipofullerene comprises a structural formula of [C60]x[C8H14O2]y, wherein x is greater than or equal to 1, and wherein y is greater than or equal to 1.

13. The chemical composition of claim 1, wherein the chemical composition is incorporated in a pharmaceutical composition.

14. A method of promoting hair growth and/or inhibiting hair loss, comprising:
topically administering to a subject an effective amount of a chemical compound comprising a lipofullerene;
topically administering to the subject an activator; and
promoting hair growth and/or inhibiting hair loss.

15. The method of claim 14, further comprising affecting the cellular permeability using the activator.

16. The method of claim 14, further comprising binding at least a portion of the activator to the lipofullerene to convey the lipofullerene intracellularly.

17. The method of claim 14, further comprising increasing production of ATP.

18. The method of claim 14, wherein the chemical compound and the activator are administered to the subject separately.

19. The method of claim 14, wherein the activator comprises dimethyl sulfoxide.

20. The method of claim 14, further comprising increasing production of ATP in follicular cells.
